(19) United States
(12) Patent Application Publication
(10) Pub. No.: US 2011/0003335 A1
(43) Pub. Date: Jan. 6, 2011

(54) METHODS FOR PROGNOSING THE STATUS OF TUMOR PATIENTS

(75) Inventors: Martin Thurnher, Aldrans (AT); Reinhold Ramoner, Innsbruck-Hoetting (AT)

Correspondence Address: COLLARD & ROE, P.C., 1077 NORTHERN BOULEVARD, ROSLYN, NY, 11576, US

(73) Assignee: Martin Thurnher, Aldrans (AT)

(21) Appl. No.: 12/735,949

(22) PCT Filed: Feb. 26, 2009
(86) PCT No.: PCT/EP2009/052305

§ 371 Date: Aug. 27, 2010

(30) Foreign Application Priority Data
Feb. 28, 2008 (EP) 08450024.8
May 15, 2008 (EP) 08450080.0

(51) Int. Cl. C12P 21/00; G01N 33/68; C07K 16/12; C07K 16/14

(52) U.S. Cl. 435/69.6; 436/86; 435/70.1; 530/388.15

(57) ABSTRACT

A method for prognosing the status of a tumor patient is provided, wherein the level of antibodies against Saccharomyces cerevisiae, Candida sp., especially Candida albicans, Aspergillus fumigatus or Klebsiella pneumoniae is determined in said the patient, and prognosing the status of the patient upon the level of these antibodies determined in the patient by determining a better status for a patient with a lower level of these antibodies compared to the average level or by determining a worse status for a patient with a higher level of these antibodies compared to the average level.

The present invention relates to methods for prognosing the status of tumor patients.

Renal cell carcinoma (RCC), which arises from the renal epithelium, is the major histologic subtype of human kidney cancer and accounts for 85% of renal cancers and for 2-3% of all cancers in adults. Approximately 30.000 to 40.000 new cases of RCC are diagnosed in the United States each year resulting in more than 12.000 deaths per year. At diagnosis a quarter of RCC patients present with advanced disease and one third of the patients with organ-confined tumors at the time of resection will develop metastatic disease. Median survival for patients with metastatic disease is about 13 months.

The physiological drug resistance of renal epithelial cells may be responsible for the low response rates of RCC to chemotherapy. Instead frequent tumor infiltration by leukocytes, occasional spontaneous tumor regression and tumor regression that may occur in the context of cytokine therapy support the value of immunotherapy including therapeutic anti-tumor vaccination. Since RCC is a highly vascularised tumor, the pathways of angiogenesis are additional targets of RCC therapy.

Defining the prognosis of RCC is important for both therapeutic decision-making and counselling patients. Several prognostic factors that correlate with overall survival in patients with metastatic RCC have previously been identified. These factors include Karnofsky performance status, serum lactate dehydrogenase (LDH), corrected serum calcium, haemoglobin, Fuhrman nuclear grade and serum cytokines. In addition, recently interleukin-4 promoter polymorphisms were described as a genetic prognostic factor for survival in metastatic RCC.

A recent Italian case-control study by Bravi et al. reported that a diet rich in bread and refined cereals may have an unfavourable influence on RCC (Bravi et al, 2007). Among several possibilities, one interpretation of the finding by Bravi et al. is that the unfavourable influence of bread on RCC reflects an underlying intolerance of wheat, gluten-containing cereals or other bread ingredients. Broken immune tolerance can cause inflammatory and immune responses to food antigens. Intolerance to wheat gluten, for instance, can lead to the development of celiac disease. Gluten-derived peptides presented on distinct MHC class II initiate CD4+ T helper (TH) cell responses followed by B cell activation and antibody production. In addition to autoantibodies against the enzyme tissue transglutaminase (TG2), IgG and IgA antibodies against gliadin, the alcohol-soluble protein fraction of gluten, are disease-specific markers.

The knowledge of the status or prognosis of a tumor patient is detrimental for the specific definition and set-up for the treatment as well as for defining the optimum treatment regimen. Patients with a good prognosis receive a less stringent treatment; patients with a poor prognosis have to receive a more stringent therapy with sometimes life threatening doses of medicaments.

In US 2004/166546 A1 methods and means for diagnosing Candida infections are disclosed. Matsuzaki et al., 2007 report Interleukin-17 as an effector molecule of innate and acquired immunity against infections. Atzpodien et al., 2003 disclose a metastatic renal carcinoma prognostic system. Naugler et al., 2008 report about the role of interleukin-6 in immunity, inflammation and cancer. Frankenberger et al., 2007 review immune suppression in renal cell carcinoma. It is therefore an object of the present invention to provide markers for the status and/or prognosis of a tumor patient.

Therefore, the present invention provides a method for prognosing the status of a tumor patient, characterised in that the level of antibodies against Saccharomyces cerevisiae, Candida sp., especially Candida albicans, Aspergillus fumigatus or Klebsiella pneumoniae is determined in said patient, and prognosing the status of the patient upon the level of these antibodies determined in the patient by determining a better status for a patient with a lower level of these antibodies compared to the average level or by determining a worse status for a patient with a higher level of these antibodies compared to the average level.

During the research for the present invention it surprisingly turned out that antibodies against Saccharomyces cerevisiae, Candida sp., especially Candida albicans, Aspergillus fumigatus or Klebsiella pneumoniae directly correlate to the prognosis/status of a patient, i.e. the lower the titer of these antibodies is, the better the status or prognosis of the patient. Saccharomyces cerevisiae, Candida sp., especially Candida albicans, Aspergillus fumigatus and Klebsiella pneumoniae share the common feature in connection with the present invention that they are microorganisms which elicit a T_{H}17-type immune response.

This was even more surprising in view of the teachings of Bravi et al. according to which intolerance of wheat is the reason for unfavourable influence of bread on RCC. In fact, the present inventors initially examined the possible relationship between humoral immune responses against bread components and an unfavourable clinical course of RCC. For this purpose, an ELISA-based screening for food-specific IgG in sera of patients with metastatic RCC was performed. During this research, however, it turned out that RCC patients with elevated serum levels of IgG antibodies against S. cerevisiae, commonly known as baker's yeast and yet another bread component, have an unfavourable clinical course. It could be shown that serum levels of IgG against S. cerevisiae predict survival in patients with metastatic RCC (Ramoner et al., 2008). The data therefore confirmed not cereals but baker's yeast being the critical component of bread that may cause immune deviation and impaired immunosurveillance in predisposed RCC patients. Since this kind of antibodies seem to be strikingly characteristic for a T_{H}17-type differentiation, it is clear that enhanced T_{H}17-type development caused by these microorganisms is not only relevant for RCC, but also for all other tumor diseases, solid and hematological cancers. Preferred tumor patients according to the present invention are patients having kidney cancer, prostate cancer, gastrointestinal cancer, ovarian cancer, breast cancer, head and neck cancer, lung cancer, non-small cell lung cancer, cancer of the nervous system, stomach cancer, liver cancer, pancreatic cancer, genital-urinary cancer, colorectal cancer, rectal cancer, bladder cancer, leukemia (especially Acute lymphoblastic leukemia (ALL), Acute myelogenous leukemia (AML), Chronic myelogenous leukemia (CML), Chronic lymphocytic leukemia (CLL) or Hairy cell leukemia), lymphoma (especially Hodgkin's disease (four subtypes) or Non-Hodgkin lymphoma (NHL, many subtypes)) or Multiple myeloma (MM). Patients with kidney cancer, prostate cancer and haematological malignancies (=haematological cancers) are specifically preferred.

Although it has been known that humoral immune responses against food components can also amount to an increased production of IgG against S. cerevisiae, commonly known as baker's or brewer's yeast, relevance of such common antibodies for e.g. tumor patients has been completely unknown. Unusually high levels of serum IgG against S. cerevisiae have been reported to be associated with inflammatory bowel diseases and in particular with Crohn's disease (WO 2007/014238 A; Forcione et al., 2004). Serum IgG against S. cerevisiae

have also been detected in patients with celiac disease or patients with ulcerative colitis (Barta et al., 2003). However, a correlation between ASCA and (the risk for) tumours, such as solid tumors or lymphomas, or between ASCA and the development of a tumor disease (i.e. as a prognostic marker) has not been disclosed or suggested in the prior art.

More in detail, the present invention relates to a method for prognosing the status of a tumor patient, wherein the level of antibodies against Saccharomyces cerevisiae, Candida sp., especially Candida albicans, Aspergillus fumigatus or Klebsiella pneumoniae is determined in said patient, and prognosing the status of the patient upon the level of these antibodies determined in the patient by determining a better prognosis for a patient with a lower level of these antibodies compared to the average level or by determining a worse prognosis for a patient with a higher level of these antibodies compared to the average level. For example, if detected by ELISA, serum levels (OD) of 0.074-1.158 (median 0.200) were measured for Saccharomyces IgG. The higher this level is, the worse the prognosis. Although there is no threshold value from which “poor prognosis” status, any level above the median is in any way bad and could e.g. exclude the patient from immune therapy, so that e.g. only “targeted therapy” with tyrosine kinase inhibitors is suited.

Although antibody detection in blood-derived patient samples (blood, serum, plasma, etc.) is preferred, the antibodies may also be detected in other antibody containing body fluids or tissue (biopsy material, etc.).

The level of antibodies against Saccharomyces cerevisiae, Candida sp., especially Candida albicans, Aspergillus fumigatus or Klebsiella pneumoniae may be determined by any method available in the art, such as ELISA-based methods, mass spectroscopy, etc. (see e.g. Current Protocols in Immunology; J. Wiley and Sons, Inc., 1991-2007).

Saccharomyces cerevisiae, Candida sp., especially Candida albicans, Aspergillus fumigatus or Klebsiella pneumoniae elicit a T_{H}17-type (biased) immune response especially in humans (e.g. Bozza et al., 2008; Ye et al, 2001). The present invention is also based on the fact that T_{H}17-type immune responses have negative impact on tumor patients (Langowski et al., 2006 and 2007). The nature of T_{H}17-type immune responses is reviewed in the prior art e.g. in Steinman, 2007 and Acosta-Rodriguez et al., 2007 as well as in LeibundGut-Landmann et al., 2007.

Directly connected with the method described above is the need for providing antibodies against Saccharomyces cerevisiae, Candida sp., especially Candida albicans, Aspergillus fumigatus or Klebsiella pneumoniae which correlate in their ability to elicit a T_{H}17-type immune response. Antibodies against these microorganisms are only available either as polyclonal antibodies from human sera (e.g. from patients (donors) with significantly enhanced level for these antibodies (such as patients with Crohn's disease) or as monoclonal antibodies against specific antigens of these microorganisms. There is, however, a need for truly human (not only humanised) antibodies against these microorganisms, specifically monoclonal human antibodies against Saccharomyces cerevisiae, Candida sp., especially Candida albicans, Aspergillus fumigatus or Klebsiella pneumoniae. Therefore, the present invention also provides a method for producing antibodies against Saccharomyces cerevisiae, Candida sp., especially Candida albicans, Aspergillus fumigatus or Klebsiella pneumoniae with the following steps:
isolation of B cells producing antibodies against Saccharomyces cerevisiae, Candida sp., especially Candida albicans, Aspergillus fumigatus or Klebsiella pneumoniae;
cultivation of the isolated B cells so as to obtain a B cell culture;
inducing antibody production of said B cells in said B cell culture; and
isolating the antibodies against Saccharomyces cerevisiae, Candida sp., especially Candida albicans, Aspergillus fumigatus or Klebsiella pneumoniae from the B cell culture.

Methods for making human monoclonal antibodies from memory B cells are—in principle—known in the art, e.g. from Traggiai et al., 2004, however, the specific need for these antibodies was not given before the present invention was made, because the antibodies according to the present invention are specifically suited to determine the tumor patient status disclosed. Other antibodies (either polyclonal antibodies from Crohn's sera or humanised monoclonal antibodies) would not fulfil the needs of the present invention as satisfactorily as the antibodies of the present invention, especially in view of the need for large scale diagnosis on a reproducible level. For this a constant level of quality is required which is fulfilled best with the human monoclonal antibodies disclosed herein. The present invention therefore also relates to monoclonal human antibodies against Saccharomyces cerevisiae, Candida sp., especially Candida albicans, Aspergillus fumigatus or Klebsiella pneumoniae as such, which can be produced according to the methods disclosed herein.

In Traggiai et al. 2004, also an example for the isolation step is given: B cells are isolated immunomagnetically by CD22 microbeads; the B cells are then immortalised with EBV in the presence of CpG oligonucleotides; B cell clones are established by limited dilution and culture supernatants are screened for the presence of e.g. Saccharomyces, Klebsiella, Candida or Aspergillus IgG, i.e. by screening the supernatant with Saccharomyces, Candida or Aspergillus ELISAS.

Preferably, the cultivation is performed after infection of the B cells with Epstein-Barr virus (EBV), preferably in combination with an infection enhancing factor such as all cytokines of the T_{H}17-type family, preferably IL-21 and/or CD40 ligand, especially in combination with oligonucleotides containing a CpG motif.

Preferably, the induction is performed by addition of cytokines, especially interleukin 21 (IL-21) or, although with less efficiency, interleukin 2 (IL-2). This is e.g. described in Kuchen et al., 2007 or Ettinger et al., 2005.

According to the present invention, antibodies against Saccharomyces cerevisiae, Candida sp., especially Candida albicans, Aspergillus fumigatus or Klebsiella pneumoniae include all Ig isotypes (IgM, IgG1-4, IgA (IgE, to a less extent) which are directed against Saccharomyces cerevisiae, Candida sp., especially Candida albicans, Aspergillus fumigatus or Klebsiella pneumoniae in patient fluids or tissues and to correlate the levels of such antibodies with the course of a disease. Furthermore, according to the present invention antibodies (all Ig isotypes) are measured which are directed against bacteria such as Klebsiella, which are known to induce T_{H}17-type immune responses in patient fluids or tissues and to correlate the levels of such antibodies with the course of a disease.

According to the present invention it is possible to use antibodies directed against Saccharomyces, Candida, Aspergillus, Klebsiella (or other T_{H}17-type-related organisms) as a prognostic indicator to predict the clinical course in tumors. Antibodies directed against Saccharomyces, Candida, Aspergillus, Klebsiella (or other T_{H}17-type-related organisms) may also be used as a serum marker of a bias of the immune system toward T_{H}17 (serum marker of T_{H}17-type bias; according to Steinman, 2007).

The present invention also relates to the use of the level of antibodies against Saccharomyces cerevisiae, Candida sp., especially Candida albicans, Aspergillus fumigatus or Klebsiella pneumoniae in a sample from a tumor patient for prognosing the status of the patient.

In determining the levels of antibodies against Saccharomyces cerevisiae, Candida sp., especially Candida albicans, Aspergillus fumigatus or Klebsiella pneumoniae in a sample of a tumor patient, kits for appropriately detecting and/or determining these antibodies can be used according to the present invention. These kits can be adapted to the need for the determination of the specific antibodies especially in lymph, blood, serum or plasma samples or biopsies of tumor patients. Accordingly, the present invention relates to the use of a kit for determining the level of antibodies against Saccharomyces cerevisiae, Candida sp., especially Candida albicans, Aspergillus fumigatus or Klebsiella pneumoniae in a sample for prognosing the status of a tumor patient.

The invention is further described by the following example and the drawing figures, yet without being restricted thereto.

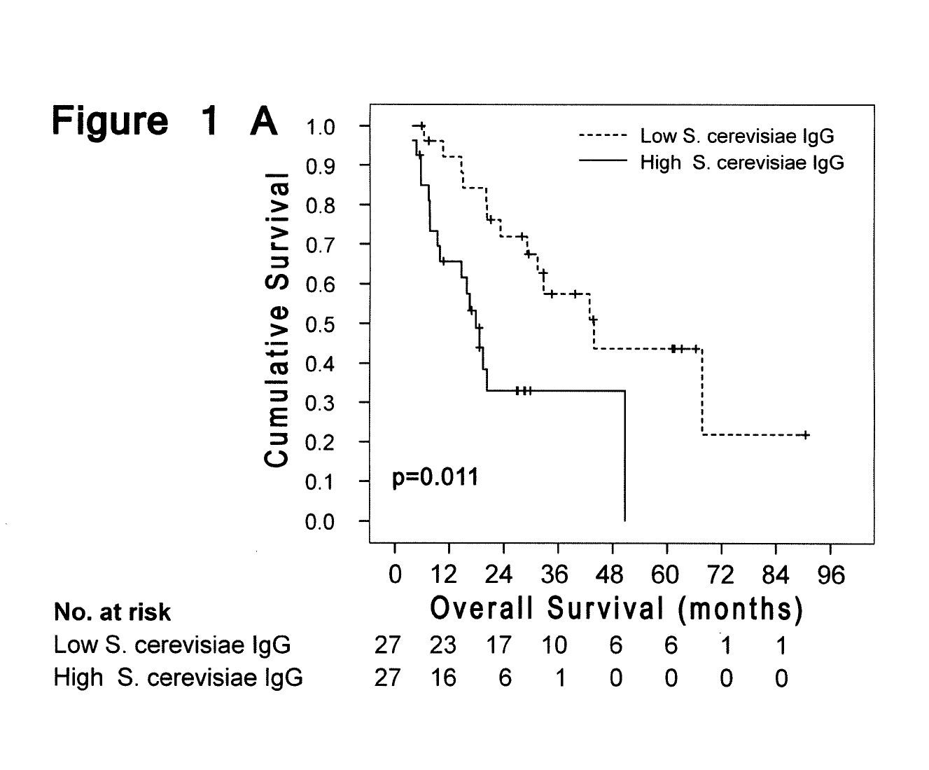

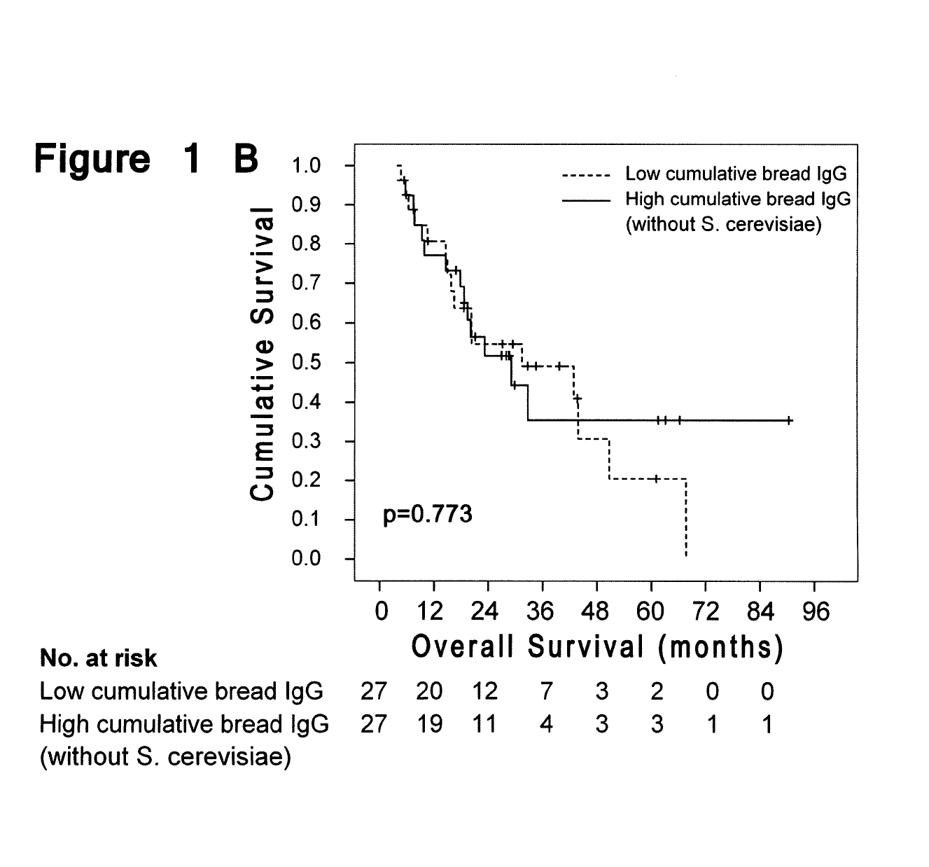

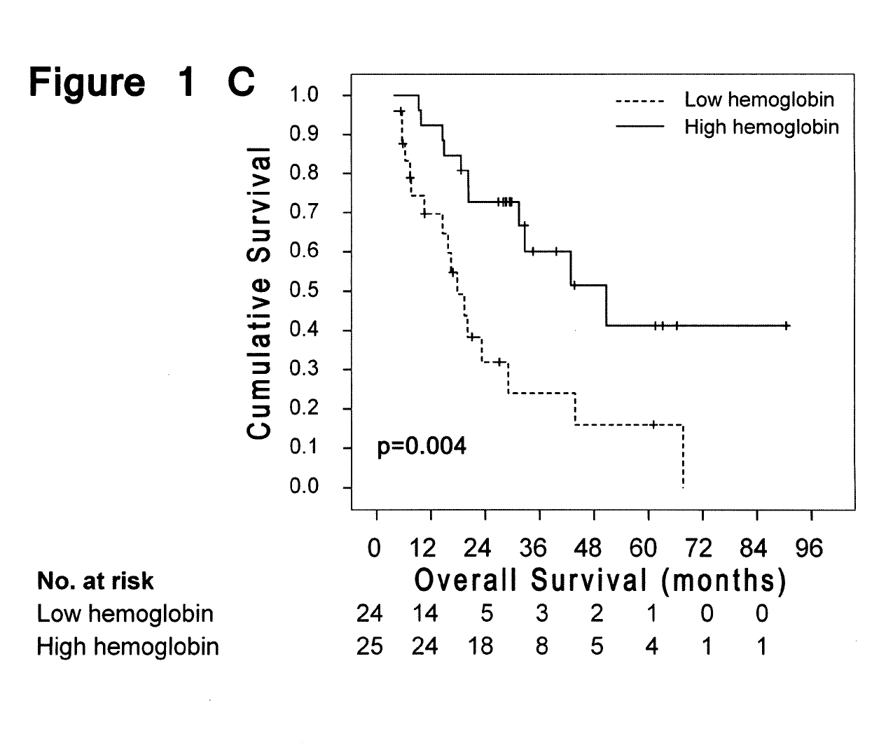

FIG. 1 shows a comparison of overall survival between patients with high or low serum levels of IgG against A) S. cerevisiae or B) cumulative bread components without S. cerevisiae; C) Comparison of overall survival between patients with high or low serum levels of hemoglobin;

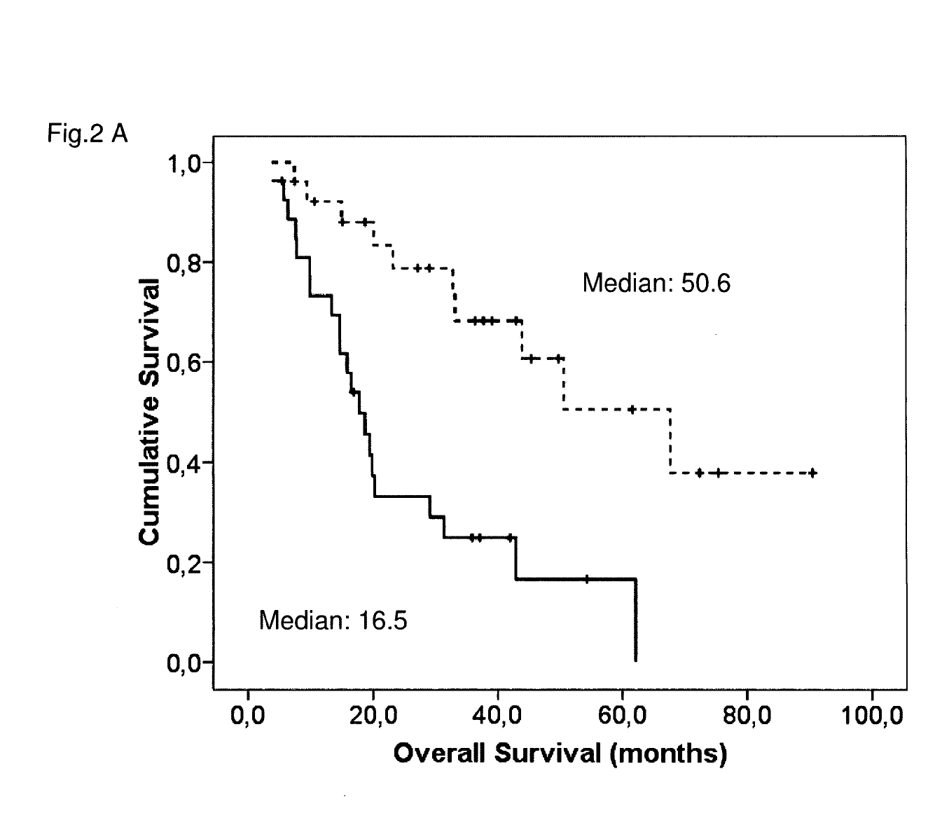

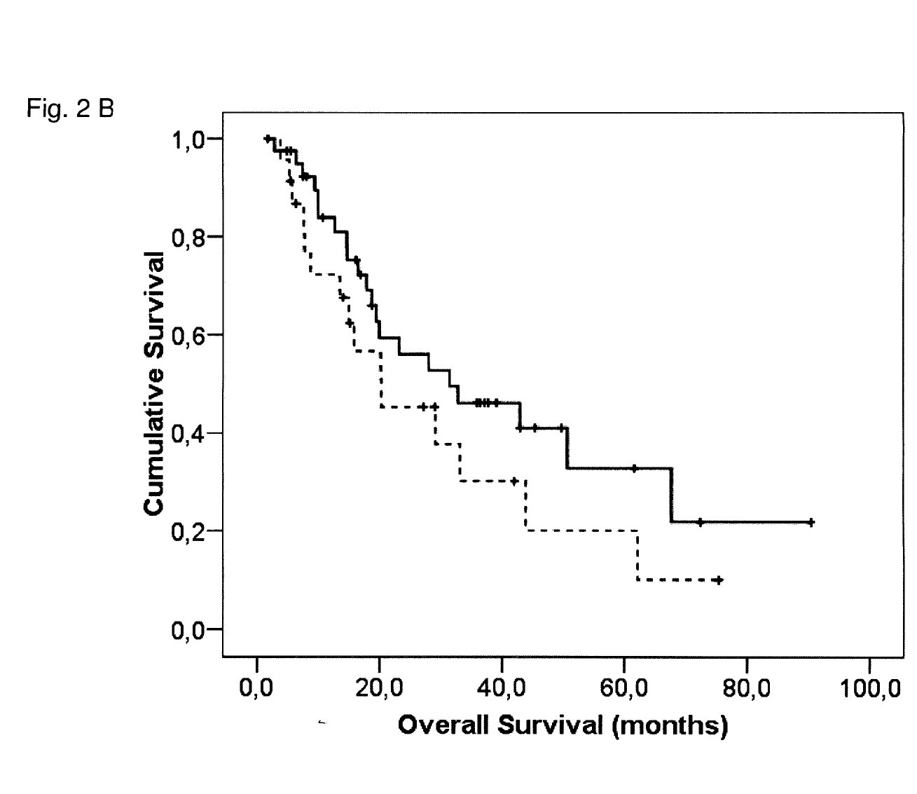

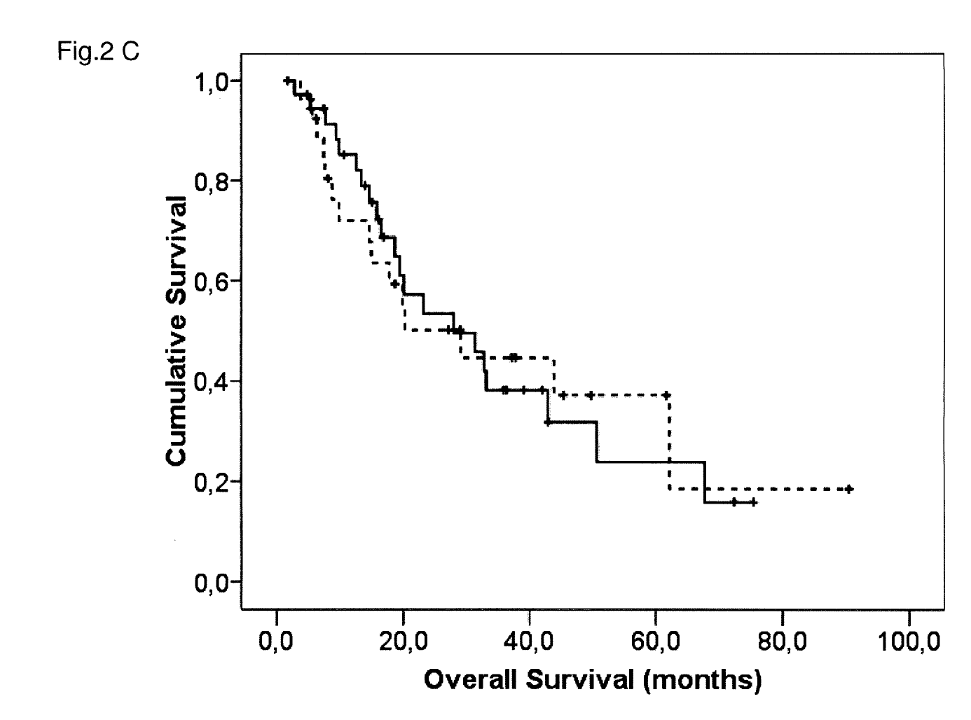

FIG. 2 shows a comparison of overall survival between metastatic RCC patients with high or low serum levels of IgG against A) Candida ssp. or B) Bordetella pertussis or C) Mycoplasma pneumoniae.

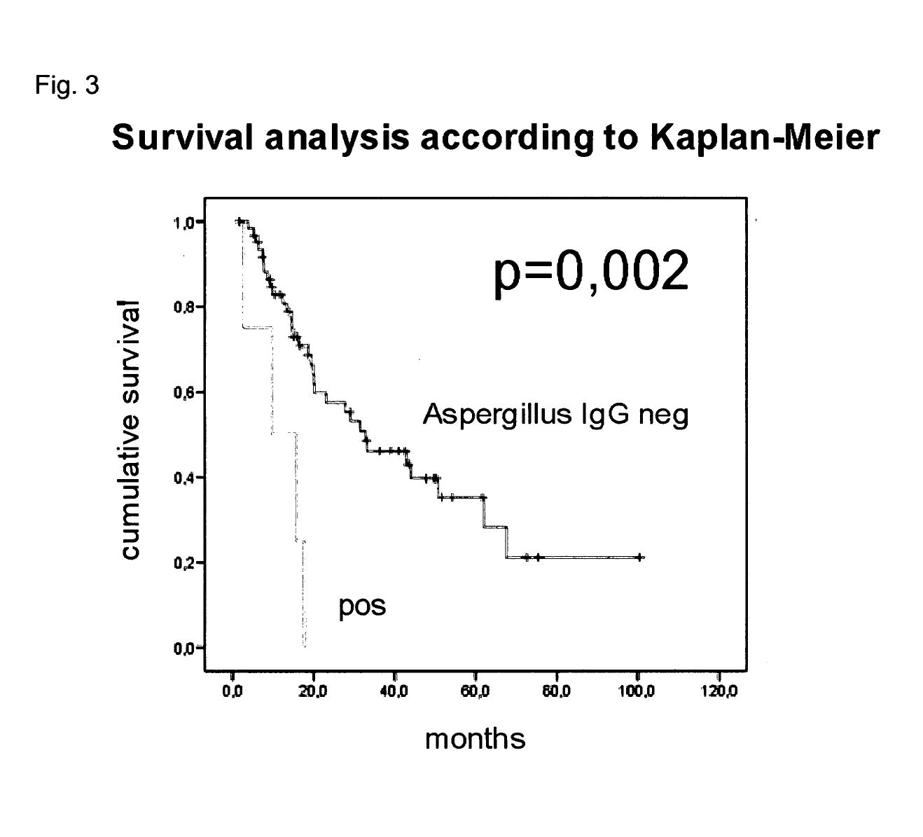

FIG. 3 shows a comparison of median survival between metastatic RCC patients being positive for Aspergillus IgG with the patients being negative for Aspergillus IgG;

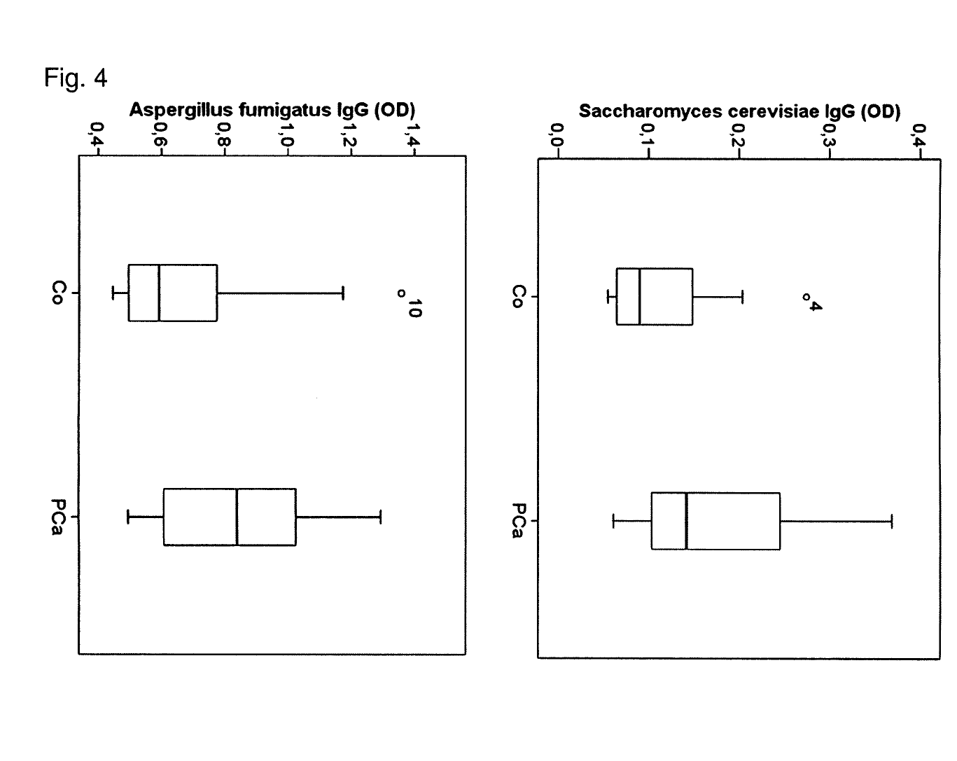

FIG. 4 shows a boxplot of serum IgG;

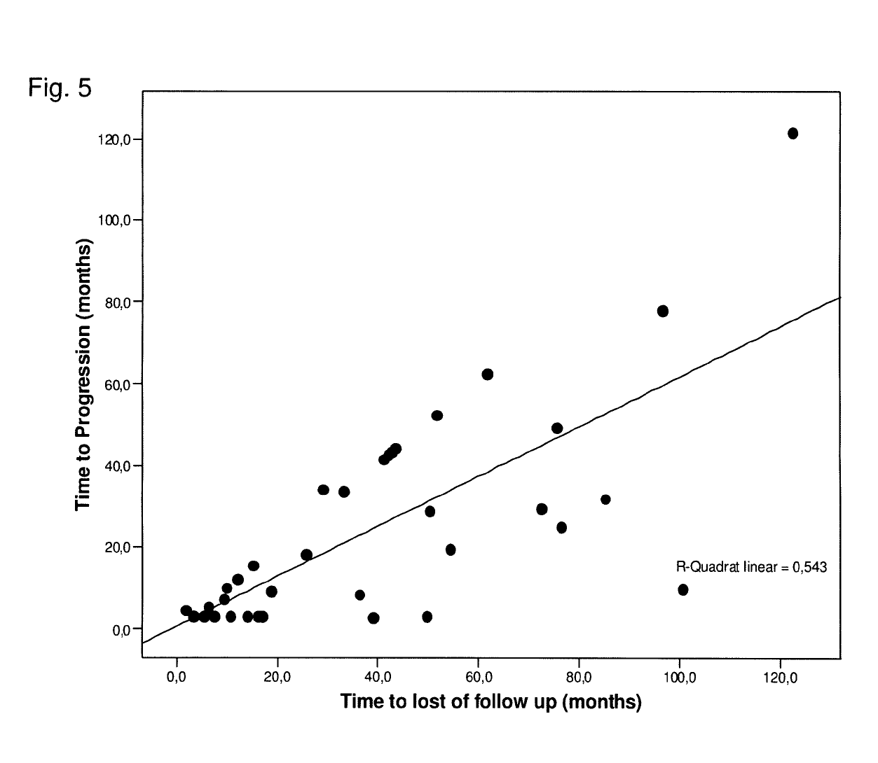

FIG. 5 shows the correlation plot of time to progression with time to lost of follow up in RCC patients;

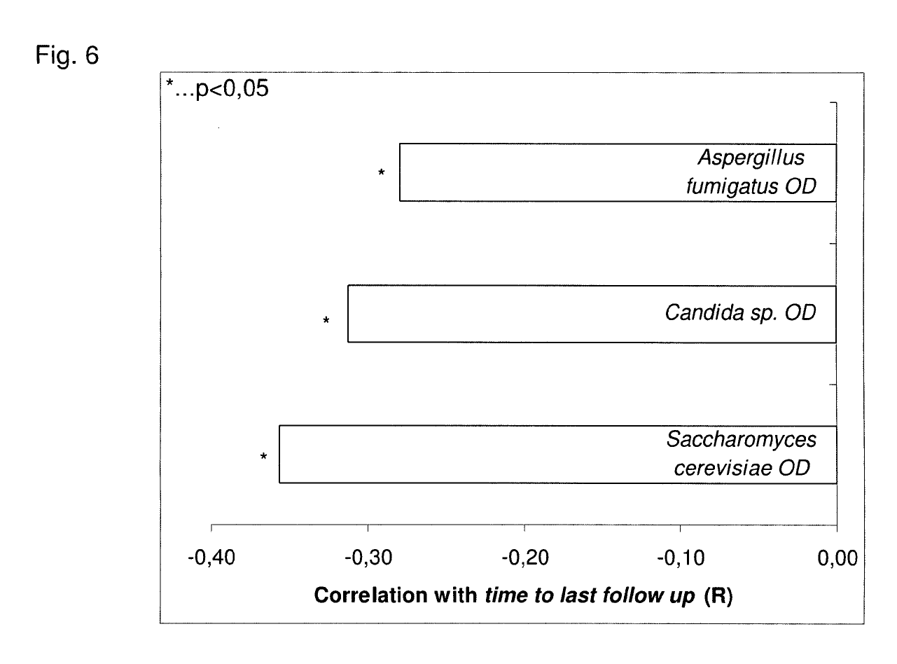

FIG. 6 shows the correlation of time to lost of follow up with anti-fungi IgG (OD);

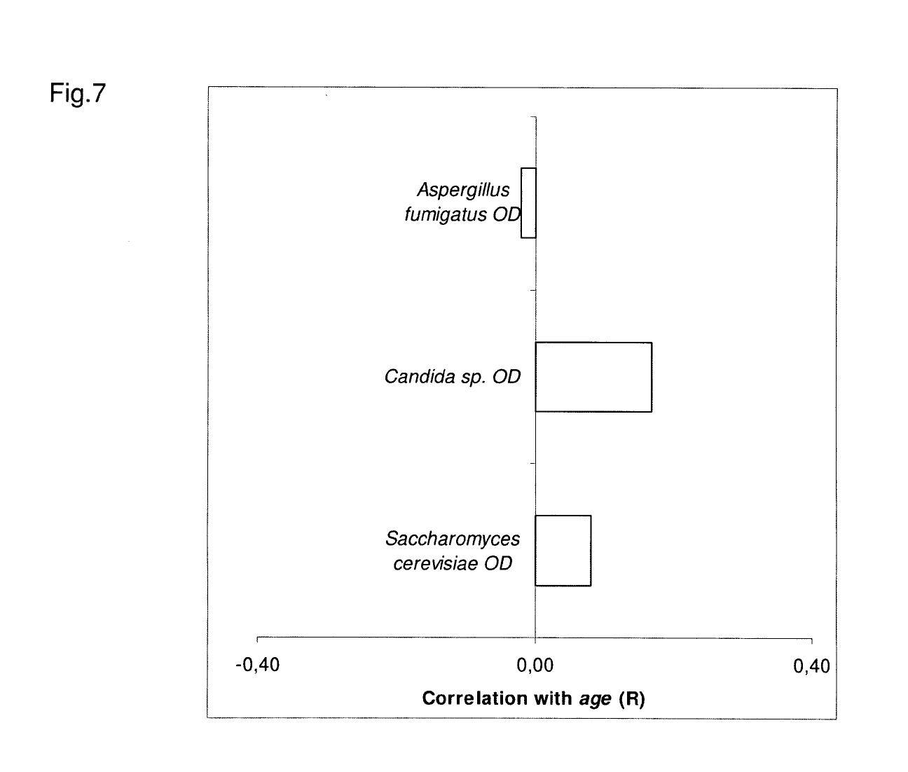

FIG. 7 shows the correlation of time to lost of follow up with age of patients.

### EXAMPLES

### Example 1

Antibodies against fungi as a prognostic indicator and a therapeutic target in cancer.

Experimental design: A commercial test was used to detect food-specific IgG directed against a panel of 113 food antigens in sera of 54 patients with metastatic RCC. Kaplan-Meier estimates were used for univariate survival analysis and differences in survival curves were assessed with the log-rank test. Multivariate survival analysis was done using a Cox regression model. Results: It was found that RCC patients with elevated serum levels of IgG antibodies against S. cerevisiae, commonly known as baker's yeast and yet another bread component, have an unfavourable clinical course. Median survival of patients with high levels of S. cerevisiae IgG was only 17.8 months, whereas median survival of patients with low S. cerevisiae IgG was 43.8 months (p=0.0022; log-rank). Multivariate survival analysis identified high levels of S. cerevisiae

IgG as a strong and independent prognostic risk factor (risk ratio 4.6, p=0.001; 95% CI 1.61-13.08).

Conclusions: These findings show that serum levels of IgG against S. cerevisiae predict survival in patients with metastatic RCC. The data suggest not cereals but baker's yeast being the critical component of bread that may cause immune deviation and impaired immunosurveillance in predisposed RCC patients.

Patients and Methods

Patients

54 RCC patients predominantly with clear-cell histology and bi-dimensionally measurable metastatic lesions were selected in a single-center study based on the availability of blood serum. No patient refused to enter the study. All patients were treated on consecutive IRB-approved dendritic cell vaccine trials, two of which have been published (Holtl et al., 2002, 2005). The primary tumor had been removed in all patients and the start of follow up was defined by the beginning of immunotherapy. Serum samples were collected prospectively prior to the onset of immunotherapy. Patients with solitary brain metastasis, other malignancies than RCC within the last 5 years, treatment with immunosuppressive drugs, other immunotherapies or chemotherapies within 4 weeks prior to treatment start, pregnancy or lactation, presence of acute or chronic infections, HIV or viral hepatitis or a Karnofsky index <60 were excluded from the study. Furthermore, a computed tomography (CT) of brain, chest and abdomen and a bone scan were performed. All patients were informed about the investigative character of the study and gave their written informed consent.

Measurement of Circulating Anti-Food IgG in Patient Serum

In the FoodSCAN Totality assay, patient serum samples were subjected to an enzyme-linked immunosorbent assay (ELISA) from YORKTEST Laboratories Ltd., Mils, Tirol, Austria, which uses microtiter plates coated with a panel of 113 different food antigens (Atkinson et al., 2004). Anti-food antibodies present in the patient's serum sample bind to the immobilized food antigen on the plate. A horseradish peroxidase-conjugated goat anti-human IgG was used to detect plate-bound food-specific patient IgG. Peroxidase-catalyzed colour development using TMB as a substrate was subsequently measured using a visible light spectro-photometer (Molecular Devices E-Max Microtiter Plate Reader). Optical density (OD) at 450 nm is directly related to the concentration of anti-food IgG antibodies present in the sample. Test results were scored as positive or negative according to the manufacturer's cut-off definitions. In addition, the median IgG level was calculated (Table 2) to generate a high and a low level group and to perform survival analysis on the two groups.

Survival Analysis

The endpoint of interest was survival time, defined as the time from treatment initiation to the date of death or the date of censure. For univariate analyses, Kaplan-Meier methodology was used to estimate survival distributions for bread components (n=5, Table 4) and clinical variables (Table 5). The relationship between survival and each of the variables was analyzed using the log-rank test.

A forward stepwise conditional Cox regression approach was used for the multivariate analyses, with 0.05 for entering and 0.1 for removing a variable in the model. Variables selected for the Cox model were age, sex, nuclear grade, serum IL-6, TNF-α, CRP, hemoglobin, lactate dehydrogenase (LDH) and S. cerevisiae IgG. The hazard ratios (risk ratios) and 95% confidence interval (CI) are reported. A two-sided p value of 0.05 or less was considered to indicate statistical significance. All calculations were carried out with SPSS software 13.0 (SPSS Inc., Chicago, Ill.).

Results

Potential food intolerances identified by serum IgG testing were analyzed in 54 patients with metastatic RCC. Table 1 summarizes general patient characteristics and Table 2 shows median baseline laboratory parameters.

TABLE 1
Patient characteristics

| Characteristic | No. | % |
|---|---|---|
| Age (years) | | |
| Median | 58.0 | |
| Range | 30-57 | |
| Sex | | |
| Female | 14 | 25.9 |
| Male | 40 | 74.1 |
| Median follow up (months) | 28.6 | |
| Range | 5.4-90.5 | |
| Pathologic stage | | |
| M0 | 5 | 9.3 |
| M1 | 49 | 90.7 |
| N0 | 29 | 53.7 |
| N1 | 14 | 25.9 |
| N2 | 9 | 16.7 |
| N3 | 2 | 3.7 |
| T1 | 9 | 16.7 |
| T2 | 14 | 25.9 |
| T3a | 15 | 27.8 |
| T3b | 14 | 25.8 |
| T4 | 1 | 1.9 |
| TX | 1 | 1.9 |
| Histologic subtype | | |
| Clear cell | 50 | 92.6 |
| Clear cell with sarcomatoid proportion | 3 | 5.6 |
| Papillary | 1 | 1.9 |

TABLE 2
Baseline laboratory parameters

| Laboratory serum parameter | Median | Range |
|---|---|---|
| Protein (g/dl) | 7.2 | 6.2-8.5 |
| Albumin (mg/dl) | 4020 | 2300-5090 |
| Hemoglobin (g/l) | 133.0 | 82-160 |
| C-reactive protein (CRP) | 0.76 | 0.0-15.9 |
| Lactate dehydrogenase (U/ml) | 181.0 | 121-404 |
| IL-6 (pg/ml) | 8.0 | 0.0-463 |
| TNF-alpha (pg/ml) | 23.0 | 0.0-274 |
| Total calcium level (mmol/l) | 2.39 | 2.0-2.8 |
| Serum IgG levels | | |
| IgG against S. cerevisae (OD) | 0.200 | 0.074-1.158 |
| IgG against gluten (OD) | 0.082 | 0.054-1.047 |
| IgG against rye (OD) | 0.073 | 0.054-0.290 |
| IgG against wheat (OD) | 0.095 | 0.053-0.980 |
| IgG against spelt (OD) | 0.090 | 0.051-1.176 |

In the total study population, 49 of 54 patients (90.7%) had positive IgG testing to an average of 4 food antigens per patient (range: 1-18). RCC patient IgG were directed against 46 of the 113 different food antigens. Table 3 shows the distribution of food intolerances within the study population.

TABLE 3
Frequencies of food-specific IgG (n = 54)

| Food | Frequency |
|---|---|
| S. cerevisiae | 29(53.7%) |
| Cow's milk | 24(44.4%) |
| Egg white | 17(31.5%) |
| Egg yolk | 10(18.5%) |
| Mussels | 10(18.5%) |
| Beef | 8(14.8%) |
| Goat's milk | 7(13.0%) |
| Spelt | 7(13.0%) |
| Wheat | 6(11.1%) |
| Millet | 5(9.3%) |
| Kiwi | 5(9.3%) |
| Garlic | 5(9.3%) |
| Pole beans | 4(7.4%) |
| Gluten | 3(5.6%) |
| Hazelnut | 3(5.6%) |
| Lamb | 3(5.6%) |
| Cabbage mix | 3(5.6%) |
| Melon mix | 3(5.6%) |
| Cashew | 3(5.6%) |
| Almond | 2(3.7%) |
| Cowberry | 2(3.7%) |
| Chili | 2(3.7%) |
| Rye | 2(3.7%) |
| Sole | 2(3.7%) |
| Coconut | 2(3.7%) |
| Orange | 2(3.7%) |
| Kidney bean | 1(1.9%) |
| Sunflower seed | 1(1.9%) |
| Corn | 1(1.9%) |
| Rice | 1(1.9%) |
| Crustaceae | 1(1.9%) |
| Withe fish mix | 1(1.9%) |
| Aubergine | 1(1.9%) |
| Cucumber | 1(1.9%) |
| Lenses | 1(1.9%) |
| Lettuce | 1(1.9%) |
| Paprika | 1(1.9%) |
| Soy beans | 1(1.9%) |
| Ginger | 1(1.9%) |
| Mustard | 1(1.9%) |
| Sesame | 1(1.9%) |
| Strawberry | 1(1.9%) |
| Red currant | 1(1.9%) |
| Cherry | 1(1.9%) |
| Plum | 1(1.9%) |
| Ananas | 1(1.9%) |

According to manufacturer's cut off

The most frequent intolerances were to S. cerevisiae

(53.7%), cow's milk (44.4%), egg white (31.5%), egg yolk (18.5%), and mussels (18.5%) and thus similar although not identical in prevalence compared to other non-cancer patient study populations (Atkinson et al., 2004).

In the univariate survival analysis, however, patients with elevated serum levels (of IgG antibodies against S. cerevisiae, commonly known as baker's or brewer's yeast and yet another bread component, had an unfavourable clinical course (FIG. 1A and Table 4).

TABLE 4
Univariate survival analysis of IgG against food antigens

| Factor | Category | N | Median Survival | 95% - Confidence Interval | P-value (log-rank) |
|---|---|---|---|---|---|
| S. cerevisiae IgG | Low | 27 | 43.8 | 25.2-62.4 | 0.011* |
| FoodSCAN Totality (OD) | High | 27 | 17.8 | 13.6-22.0 | |
| Gluten IgG | Low | 27 | 31.4 | 10.4-52.4 | 0.292 |
| | High | 27 | 29.1 | — | |
| Rye IgG | Low | 27 | 42.9 | 0.6-85.1 | 0.917 |
| | High | 27 | 29.1 | 12.7-45.5 | |
| Wheat IgG | Low | 27 | 31.4 | 5.6-57.1 | 0.721 |
| | High | 27 | 29.1 | 16.0-42.1 | |
| Spelt IgG | Low | 27 | 42.9 | 7.1-78.6 | 0.929 |
| | High | 27 | 23.2 | 11.7-34.7 | |
| Cumulative bread IgG | Low | 27 | 31.4 | 5.6-57.2 | 0.773 |
| without S. cerevisiae | High | 27 | 29.1 | 13.0-45.2 | |
| Cumulative bread IgG | Low | 27 | 42.9 | 27.2-58.6 | 0.211 |
| with S. cerevisiae | High | 27 | 20.3 | 14.6-26.1 | |
| Food IgG | not present | 5 | all censured | all censured | 0.069 |
| | present | 49 | 23.2 | 11.7-34.6 | |
| All patients | | 54 | 31.4 | 16.3-46.5 | |

* Bonferroni adjusted significance level for bread components; OD, optical density;

Median survival of patients with high levels of S. cerevisiae IgG was only 17.8 months, whereas median survival of patients with low S. cerevisiae IgG was 43.8 months (p=0.0022; log-rank). The difference remains significant after a error adjustment according to Bonferroni (p=0.011).

In contrast to IgG against S. cerevisiae,

IgG antibodies against other bread components (gluten, rye, wheat and spelt) did not show a significant influence on patient survival although high levels were always accompanied by reduced survival (Table 4). Even cumulative values of IgG to all 4 bread components excluding S. cerevisiae IgG did not show a correlation with survival (FIG. 1B) with almost identical median survival times for the high and low IgG level group (29.1 and 31.4 months; Table 4). When cumulative values of IgG to all bread components that included S. cerevisiae

IgG were analyzed, median survival for the high and low IgG group was 20.3 and 42.9 months, respectively (p=0.211; Table 4) showing that S. cerevisiae IgG and not IgG to other bread components correlate with patient survival. The well-established prognostic factor hemoglobin (Hb) showed the expected correlation with patient overall survival (FIG. 1C).

Of all 54 patients tested, 5 patients were completely negative for IgG to any of the 113 food antigens. Intriguingly, all 5 patients were alive at the time of analysis and absence of IgG to food antigens showed a tendency to correlate with prolonged survival (p=0.069) (Table 4).

Several serum markers have been established as prognostic risk factors in RCC. In the present study group hemoglobin (p=0.004), IL-6 (p=0.042) as well as TNF-α (p=0.032) correlated with patient survival and LDH showed a tendency to correlate with patient survival (p=0.092) (Table 5).

TABLE 5
Univariate survival analysis of demographic and clinical variables

| Factor | Category | N | Median Survival | 95% - Confidence Interval | P-value (log-rank) |
|---|---|---|---|---|---|
| Age | <=58 | 27 | 29.1 | 14.4 - 43.8 | 0.808 |
| | >58 | 27 | 31.4 | 1.9 - 60.9 | |
| Sex | Female | 14 | 32.7 | 10.2 - 55.2 | 0.873 |
| | Male | 40 | 23.2 | 6.6 - 39.8 | |
| Nuclear Grade | 1-2 | 29 | 31.4 | 10.6 - 52.2 | 0.186 |
| | 3-4 | 24 | 20.3 | 12.8 - 27.7 | |
| IL-6 (pg/ml) | Low | 25 | 42.9 | — | 0.042 |
| | High | 24 | 19.4 | 16.0 - 22.8 | |
| TNF-alpha(pg/ml) | Low | 24 | — | — | 0.032 |
| | High | 24 | 20.3 | 15.2 - 25.5 | |
| CRP (mg/dl) | Low | 26 | 31.4 | 20.8 - 42.0 | 0.604 |
| | High | 25 | 67.7 | 8.7.2 - 126.6 | |
| Hemoglobin (g/L) | Low | 25 | 17.8 | 12.9 - 22.7 | 0.004 |
| | High | 26 | 50.7 | 25.5 - 75.8 | |
| Lactate dehydrogenase(U/mL) | Low | 25 | 42.9 | 27.9 - 57.9 | 0.092 |
| | High | 26 | 19.4 | 12.6 - 26.2 | |

Abbreviations: IL-6, interleukin-6; TNF-alpha, tumor-necrosis-factor-alpha; CRP, C-reactive protein; ASCA, anti-S. cerevisiaeantibodies;

The variables included in the multivariate analysis were S. cerevisiae IgG plus the variables shown in Table 5. Using a significance level of 0.05 for entering and 0.1 for removing a variable in a forward stepwise analysis, high levels of S. cerevisiae

IgG emerged as the strongest independent prognostic risk factor (risk ratio: 4.6, p=0.001; 95% CI 1.61-13.08). The two other variables that were retained, were TNF-α (risk ratio: 1.02, p=0.018; CI 1.01-1.04) and hemoglobin (risk ratio: 0.95 p=0.0001; CI 0.93-0.98) (Table 6).

TABLE 6
Multivariate survival analysis

| Factor | 95% CI | Risk Ratio | P-value |
|---|---|---|---|
| S. cerevisiae IgG | 1.61 to 13.08 | 4.6 | 0.001 |
| TNF-alpha | 1.01 to 1.04 | 1.02 | 0.018 |
| Hemoglobin | 0.93 to 0.98 | 0.95 | 0.0001 |

No correlation was observed between serum levels of S. cerevisiae IgG and inflammation or infection markers such as CRP (r=−0.086, p=0.546), TNF-α (r=0.004, p=0.977) or IL-6 (r=0.008, p=0.956) (Spearman-Rho). The current study included patients from two different study protocols using either autologous (Holtl et al., 2002) or allogeneic dendritic cells (Holtl et al., 2005). A current follow up revealed that the two study protocols resulted in very similar clinical outcomes with no significant difference in overall survival.

Similar results were obtained for C. albicans antibodies (FIG. 2) Measurement of serum levels of IgG directed against a mixture of Candida species (C. albicans, C. krusei, C. glabrata, C. pseudotropicalis, C. parapsilosis in equal proportion) in 65 patients with metastatic RCC revealed an even more pronounced correlation between serum IgG levels and patient survival. Median survival of patients with high levels of Candida

IgG was only 16.5 months, whereas median survival of patients with low Candida IgG was 50.6 months (p<0.05; log-rank) (FIG. 2A). In contrast, no correlation was observed between serum levels of IgG specific for Bordetella pertussis (FIG. 2B) or serum levels of IgG specific for Mycoplasma pneumoniae (FIG. 2C).

Also for Aspergillus,

an analysis of the patient survival in correlation to the presence or absence of Aspergillus IgG revealed a significantly reduced median survival for patients being positive for such IgG. Whereas patients being negative for Aspergillus

IgG showed a median survival of 32.7 months, patients positive for Aspergillus IgG had a median survival of 9.9 months only. In FIG. 3 a survival analysis according to Kaplan-Meier is given comparing the two groups of patients (Hazard Ratio 4.9 (in Cox-Regression, p=0.005)).

IgG-based humoral immunity to food antigens in human metastatic RCC was evaluated and increased serum levels of S. cerevisiae IgG identified as a strong and independent prognostic risk factor. Patients with high levels of S. cerevisiae IgG had an increased risk of curtailed survival (Table 4 and 6). A correlation between CRP, TNF-α or IL-6 with serum levels of S. cerevisiae IgG was not observed indicating that IgG antibodies to S. cerevisiae

are a stable marker, which is not regulated by inflammatory events or by infection and which is thus a useful independent prognostic indicator.

The presence of IgG antibodies is usually indicative of co-existing antigen-specific CD4+ T-cell immunity, because B cells require help from cognate CD4+ T helper (TH) cells for Ig class switching from IgM to IgG. The presence of IgG antibodies directed against S. cerevisiae in patient sera would thus be a predictor of the presence of CD4+ TH cells specific for S. cerevisiae. Intriguingly, CD4+ T-cell immunity against Candida albicans,

a closely related yeast, is predominantly mediated by the T_{H}17 subset of CD4+ T cells (Acosta-Rodriguez et al., 2007; LeibundGut-Landmann et al., 2007). T_{H}17 cells, besides T_{H}1 and T_{H}2 cells, represent a third subset of polarized effector T cells. T_{H}1 cells produce interferon-γ (IFN-γ) and confer immunity to viruses, intracellular bacteria and protozoan parasites. IFN-γ producing T_{H}1 cells are also considered crucial for antitumor immunity. In contrast, T_{H}2 cells making IL-4, IL-5 and IL-13 promote immunity against metazoan parasites. Immunopathology is often associated with T_{H}2 cells since dysregulated T_{H}2 cytokine production enhances IgE production, mucus secretion and eosinophilia thus favouring the development of allergic diseases such as asthma, allergic rhinitis, and atopic dermatitis.

T_{H}17 cells characterized by the production of IL-17 and other cytokines appear to be required for resistance to infection by extracellular bacteria such as Klebsiella pneumoniae (Ye et al., 2001) as well as by fungi such as Candida albicans (Acosta-Rodriguez et al., 2007; LeibundGut-Landmann et al., 2007). Consistent with the assumption that serum IgG antibodies against S. cerevisiae reflect T_{H}17 differentiation, T_{H}17 cytokine family members were shown to critically regulate inflammatory bowel diseases, such as Crohn's disease, where levels of IgG against S. cerevisiae are frequently increased (Barta et al., 2003; Bossuyt et al., 2006; Peeters et al., 2001), and T_{H}17 T cells have been implicated in gut inflammation and destruction.

There is increasing evidence that T_{H}1 (IFN-γ) and T_{H}17 (IL-17) may be reciprocal in terms of function and that T_{H}17 development is likely to occur at the expense of T_{H}1, particularly because IL-23, which drives IL-17 producing TH17 cells, and IL-12, which promotes IFN-γ producing T_{H}1 cells, share and thus compete for the common p40 subunit that heterodimerizes with p35 to form IL-12 or with p19 for IL-23 generation (Steinman, 2007). Enhanced T_{H}17 development, however, may favour tumor development and progression (Langowski et al., 2006 and 2007). A role for IL-23 in promoting tumor incidence and growth was also described. Moreover, IL-23 stimulated angiogenesis and antagonized IL-12 and IFN-γ, both of which are crucial effector cytokines of anti-tumor immune responses.

Consistent with the notion that yeast-dependent T_{H}17 bias are associated with an unfavourable clinical course of cancer, an early immunotherapy trial, which used an aggregated autologous tumor antigen combined with Candida albicans antigens as a non-specific adjuvant for the treatment of metastatic RCC failed to demonstrate clinical activity (Fowler, 1986). Conversely, nine of 14 RCC patients receiving the Candida-containing tumor vaccine had disease progression manifested by the appearance of previously undetected metastases during the first 3 months. It is well possible that anti-Candida vaccination had increased T_{H}17 bias in these patients and thereby promoted tumor progression.

Taken together, IgG antibodies directed against S. cerevisiae may be a serum marker of T_{H}17 differentiation and the prognostic significance of such IgG antibodies in cancer patients may be due to the impaired immunosurveillance, which is a consequence of the immune deviating effects of T_{H}17 cytokines that convert tumor-suppressing effector T cells into tumor-promoters.

The formation of large amounts of immune complexes consisting of food antigens and specific IgG antibodies on a daily basis may generally disturb immunosurveillance. Antigen-antibody complexes have been shown to inhibit IL-12 production and to induce IL-6 and prostaglandins thereby generating a milieu, which enhances T_{H}17 differentiation. In line with the assumption that immune complexes cause immunosuppression or immune deviation and thus disturb cancer immunosurveillance is the intriguing although preliminary finding in our study that the 5 patients, who had no detectable IgG against the whole panel of 113 food antigens, were all alive at the time of analysis and absence of IgG to food antigens showed a tendency to correlate with prolonged survival (p=0.069) (Table 4). Moreover, one of these 5 patients had a complete remission of all metastases during a recent immunotherapy trial with dendritic cells and is still tumor free after 2 years.

In conclusion, the study according to the present invention identifies S. cerevisiae as the critical component of bread, which has recently been shown to have an unfavourable role in the development of RCC.

### Example 2

Prostate Carcinoma

Prostate cancer (PCa) is the sixth most common cancer representing about 29 percent of all malignancies diagnosed in men. Almost 180.000 cases will be diagnosed in 2009 in the United States and about 28.000 deaths will occur. It is the second leading cause of cancer death in American men. One in six American men will be diagnosed with PCa. Reliable prognostic factors for PCa according to World Health Organization (WHO) Recommendations are TNM stage, histological grade (Gleason), surgical margin status preoperative and PSA serum level.

A comparative analysis of serum levels of IgG against fungi in patients with histologically confirmed PCa and patients with elevated PSA (>0.2 ng/ml) but no detectable PCa was therefore performed to emphasize the prognostic relevance of serum levels of IgG against fungi in patients with solid cancers.

Patients and Methods

Patients

In a retrospective single-center study, 30 patients were selected based on a PSA (prostate specific antigen) value of 0.2 ng/ml. In 15 of these patients subsequent biopsy revealed histologically confirmed PCa. Maximum PSA velocity was ≦0.39 ng/ml/year. Follow up was 3.01 years. These patients had no other carcinomas or other metabolic diseases. The other 15 patients, who also presented with a PSA value of 0.2 ng/ml but had no detectable carcinoma, served as a control group (Co). All patients and donors provided written informed consent to the storage and use of their plasma samples, which were obtained before biopsy and thus before pathohistological examination and carcinoma diagnosis.

Measurement of IgG Directed Against Fungi

Using the Sentimun CSA array, patient heparin plasma samples were subjected to an enzyme-linked immunosorbent assay (ELISA), which uses microtiter plates coated with lysates (=extracts) from Saccharomyces cerevisiae or Aspergillus fumigatus. Anti-fungi antibodies present in the patient's plasma sample bind to the immobilized fungal antigen on the plate. A horseradish peroxidase-conjugated goat anti-human IgG was then used to detect plate-bound fungi-specific patient IgG. Peroxidase-catalyzed colour development using TMB as a substrate was subsequently measured using a visible light spectrophotometer (Molecular Devices E-Max Microtiter Plate Reader). Optical density (OD) at 450 nm is directly related to the concentration of anti-yeast or anti-mold IgG antibodies present in the sample.

Statistical Methods

Normal distribution of plasma OD to yeasts and molds was tested using Shapiro-Wilk test. All groups were significant (p<0.01), therefore Mann-Whitney U test was used for group comparisons. All calculations were carried out with SPSS software 15.0 (SPSS Inc., Chicago, Ill., USA).

Results

Median serum levels of IgG against Saccharomyces and Aspergillus are both elevated in PCa as compared to the control group (36.5% and 29.4% increase respectively) (Table 7 and FIG. 4). Saccharomyces serum IgG is detected at a median level of OD=0.090 in control patients, whereas patients with histologically confirmed PCa have significantly elevated serum titers (OD=0.142, p=0.028 one-sided Mann-Whitney U). Likewise, IgG against Aspergillus are significantly elevated in the PCa group (median OD=0.839) as compared to the control group (median OD=0.592, p=0.035, one-sided Mann-Whitney U).

TABLE 7
Optical densities (OD) for Saccharomyces and Aspergil-
lus IgG

| | | OD | OD |
|---|---|---|---|
| | | Saccharomyces | Aspergillus |
| Control group | standard deviation | 0,120 | 0,269 |
| | Median | 0,090 | 0,592 |
| | Mean | 0,134 | 0,700 |
| Prostate carcinoma | tandard deviation | 0,161 | 0,489 |
| | Median | 0,142 | 0,839 |
| | Mean | 0,202 | 0,926 |

The results of example 2 show that serum IgG against yeasts and molds are significantly elevated in PCa patients as compared to a control group with elevated PSA but no carcinoma and therefore conclude that the prognostic relevance of serum IgG against fungi originally observed in renal cell carcinoma (example 1) is also valid in PCa.

Measurement of serum IgG specific for yeasts and molds in renal carcinoma was performed on a group of patients with metastatic disease and the present results indicated that high serum levels correlated with shortened survival (example 1). Thus, serum IgG against yeasts and molds have prognostic relevance in metastatic renal cancer and can be used to prognose the clinical course of the metastatic disease.

In example 2, patients with prostatic disease were examined and a group of PCa patients was compared with a group of patients with elevated PSA but no detectable PCa. The significantly elevated serum levels of IgG against yeasts and molds in the carcinoma group confirms the prognostic relevance of these antibodies and indicates that they are useful to prognose the clinical course of prostate cancer patients.

### Example 3

Haematological Malignancies

In contrast to carcinomas, haematological malignancies are the types of cancer that affect blood, bone marrow, and lymph nodes. Normal haematopoiesis is dependent on intricately regulated signalling cascades that are mediated by cytokines and their receptors. Orderly function of these pathways leads to the generation of appropriate constellation of hematopoietic cells, and their abnormal activation results in neoplastic transformation, impaired apoptosis, and uncontrolled proliferation. Chromosomal translocations are a common cause of these diseases, while this is uncommon in solid tumors.

Patients with hematologic malignancies are at substantial risk of developing invasive fungal infections that are associated with substantial morbidity and mortality. Therefore, there is a need to define patient groups at greatest risk of invasive fungal infections and, when appropriate, to initiate effective antifungal prophylaxis.

The haematological malignancies include:
Leukemia:
   Acute lymphoblastic leukemia (ALL)
   Acute myelogenous leukemia (AML)
   Chronic myelogenous leukemia (CML)
   Chronic lymphocytic leukemia (CLL)
   Hairy cell leukemia
Lymphoma:
   Hodgkin's disease (four subtypes)
   Non-Hodgkin lymphoma (NHL, many subtypes)
Multiple myeloma (MM)

Patients and Methods

Patients

42% (21) of patients were female and 58% (29) were male with a median age at diagnosis of 52.2 years (minimum 20.3, maximum 82.5, standard deviation 15.2). The most common malignancies in the analyzed patient group were AML with 38% (19), MM with 20% (19) and NHL with 16% (8) (Table 8).

TABLE 8
Frequencies of hematologic malignancies analyzed

| Diagnosis | Frequency (n) | Percent |
|---|---|---|
| ALL | 7 | 14.0 |
| AML | 19 | 38.0 |
| CLL | 2 | 4.0 |
| CML | 2 | 4.0 |
| Hodgkin Lymphoma | 1 | 2.0 |
| MM | 10 | 20.0 |
| NHL | 8 | 16.0 |
| Sarcoma | 1 | 2.0 |
| Total | 50 | 100.0 |

36.7% (18) received induction chemotherapy IND, whereas 28.6% (14) of patients received sibling allogenic (SIB) stem cell transplantation (SCT). Autologous stem cell transplant (aSCT) was administered to 18.4% (9) of patients and 10.2% (5) received matched unrelated donors (MUD) stem cell transplantation. The remaining patients received HLA-mismatched unrelated donor (MMUD) stem cell transplantation and conservative therapy (Table 9).

TABLE 9
Frequencies of administered therapies

| Therapy | Frequency (n) | Percent |
|---|---|---|
| aSCT | 9 | 18.4 |
| IND | 18 | 36.7 |
| MUD | 5 | 10.2 |
| MMUD | 1 | 2.0 |
| SIB | 14 | 28.6 |
| Conservative | 2 | 4.1 |
| Total | 49 | 100.0 |
| Missing | 1 | |

The study protocol has been reviewed by the local ethics committee and has been registered by the Austrian Agency for Health and Food Safety (AGES) (ref no INS-621000-0079-002).

Measurement of IgG Directed Against Fungi

Using the Sentimun CSA array, patient EDTA plasma samples were subjected to an enzyme-linked immunosorbent assay (ELISA), which uses microtiter plates coated with lysates (=extracts) from Saccharomyces cerevisiae, a mixture of Candida species (C. albicans, C. krusei, C. glabrata, C. pseudotropicalis, C. parapsilosis in equal proportion) or Aspergillus fumigatus. Anti-fungi antibodies present in the patient's plasma sample bind to the immobilized fungal antigen on the plate. A horseradish peroxidase-conjugated goat anti-human IgG was then used to detect plate-bound fungi-specific patient IgG. Peroxidase-catalyzed color development using TMB as a substrate was subsequently measured using a visible light spectrophotometer (Molecular Devices E-Max Microtiter Plate Reader). Optical density (OD) at 450 nm is directly related to the concentration of anti-yeast or anti-mold IgG antibodies present in the sample.

Statistical Methods

The time measured from the start of therapy to progressive disease (time to progression) was correlated with the time from the start of therapy to the time of lost of follow up (time to lost of follow up) in RCC patients. In addition a series of Sperman's rank correlations of fungal IgG with time to lost of follow up and age were calculated. A two sided p-value of p<0.05 was considered as a significant result.

Results

Because of the missing data on overall survival of patients in haematological malignancies a surrogate marker was established. A strong positive correlation exists between the variable time to progression and time to lost of follow up in RCC patients (n=36, Pearson R2=0.543, p<0.0001, FIG. 5).

Subsequently, serum levels of anti-fungi IgG were correlated with the time to lost of follow up. A significant negative correlation was found between the serum levels of Aspergillus, Candida and Saccharomyces

IgG and time to lost of follow up (R=−0.28, −0.31 and −0.36, FIG. 6).

In contrast, no significant correlation was found between IgG against fungi and the age of patients (R=−0.02, 0.17 and 0.08, FIG. 7).

In example 3 the prognostic value of IgG to yeasts and molds were analyzed in different hematologic malignancies. Follow up (i.e. the time from diagnosis to lost of follow up) was chosen as a surrogate of survival. A significant negative correlation between all three anti-fungi IgG (Aspergillus, Candida, and Saccharomyces) and follow up in hematologic malignancies was found. This demonstrates, that the presence of IgG to molds and yeasts in patients with hematologic malignancies mark a negative prognosis not dependent on age as previously shown in example 1 and 2.

### REFERENCES

Acosta-Rodriguez et al., (2007) Nat Immunol 8:942-949

Acosta-Rodriguez et al., (2007) Nat Immunol 8:639-646

Atkinson et al., (2004) Gut 53:1459-1464

Atzpodien et al., (2003) Br J Cancer 88 : 348-353

Barta et al., (2003) World J Gastroenterol 9:2308-2312

Bossuyt et al., (2006) Clin Chem 52:171-181

Bozza et al., (2008) J Immunol 180: 4022-4031

Bravi et al., (2007) Int J Cancer 120:681-685

Ettinger et al., (2005) J Immunol 175: 7867-7879

Forcione et al., (2004) Gut 53: 1117-1122

Fowler, (1986) J Urol 135:22-25

Frankenberger et al., (2007) Sem Cancer Biol 17: 330-343

Holtl et al., (2005) Cancer Immunol Immunother 54:663-670

Holtl et al., (2002) Clin Cancer Res 8:3369-3376

Kuchen et al., (2007) J Immunol 179 5886-5896

Langowski et al., (2006) Nature 442: 461-465

Langowksi et al., (2007) Trends Immunol. 28: 207-212

LeibundGut-Landmann et al., (2007) Nat. Immunol. 8: 630-638

Matsuzaki et al., Mcrobiol Immunol 51: 1139-1147

Naugler et al., (2008) Trends Mol Med 14: 109-119

Peeters et al., (2001) Am J Gastroenterol 96:730-734

Ramoner et al., (2008) Cancer Immunol Immunother 57 : 1207-1214

Steinman, (2007) Nat Med 13:139-145

Traggiai et al., (2004) Nat Med 10: 871-876

Ye et al., (2001) J Exp Med 194:519-527

1. Method for prognosing the status of a tumor patient, wherein the level of antibodies against Saccharomyces cerevisiae, Candida sp., especially Candida albicans, Aspergillus fumigatus or Klebsiella pneumoniae is determined in said patient, and prognosing the status of the patient upon the level of these antibodies determined in the patient by determining a better prognosis for a patient with a lower level of these antibodies compared to the average level or by determining a worse prognosis for a patient with a higher level of these antibodies compared to the average level.

2. Method according to claim 1, wherein the tumor patient is a patient having a solid tumor or a haematological malignancy.

3. Method according to claim 2, wherein the tumor patient is a patient having kidney cancer, prostate cancer, gastrointestinal cancer, ovarian cancer, breast cancer, head and neck cancer, lung cancer, non-small cell lung cancer, cancer of the nervous system, stomach cancer, liver cancer, pancreatic cancer, genital-urinary cancer, colorectal cancer, rectal cancer, bladder cancer, leukemia (especially Acute lymphoblastic leukemia (ALL), Acute myelogenous leukemia (AML), Chronic myelogenous leukemia (CML), Chronic lymphocytic leukemia (CLL) or Hairy cell leukemia), lymphoma (especially Hodgkin's disease (four subtypes) or Non-Hodgkin lymphoma (NHL, many sub-types)) or Multiple myeloma (MM).

4. Method according to claim 2, wherein the tumor patient is a patient with kidney cancer, prostate cancer or a haematological malignancy.

5. Method according to claim 1, wherein the level of antibodies against Saccharomyces cerevisiae is determined.

6. Method according to claim 1, wherein the level of antibodies against Candida albicans is determined.

7. Method according to claim 1, wherein the level of antibodies against Aspergillus fumigatus is determined.

8. Method according to claim 1, wherein the level of antibodies against Klebsiella pneumoniae is determined.

9. Method for producing antibodies against Saccharomyces cerevisiae, Candida sp., especially Candida albicans, Aspergillus fumigatus or Klebsiella pneumoniae comprising the following steps:
isolation of B cells producing antibodies against Saccharomyces cerevisiae, Candida sp., especially Candida albicans, Aspergillus fumigatus or Klebsiella pneumoniae;
cultivation of the isolated B cells so as to obtain a B cell culture;
inducing antibody production of said B cells in said B cell culture; and
isolating the antibodies against Saccharomyces cerevisiae, Candida sp., especially Candida albicans, Aspergillus fumigatus or Klebsiella pneumoniae from the B cell culture.

10. Method according to claim 8, wherein said cultivation is performed after infection of the B cells with Epstein-Barr virus (EBV), preferably in combination with an infection enhancing factor, especially in combination with oligonucleotides containing a CpG motif.

11. Method according to claim 8, wherein said induction is performed by addition of cytokines, especially interleukin 21 (IL-21).

12. Monoclonal human antibodies against Saccharomyces cerevisiae, Candida sp., especially Candida albicans, Aspergillus fumigatus or Klebsiella pneumoniae.

13. Use of the level of antibodies against Saccharomyces cerevisiae, Candida sp., especially Candida albicans, Aspergillus fumigatus or Klebsiella pneumoniae in a sample from a tumor patient for prognosing the status of the patient.

14. Use of a kit for determining the level of antibodies against Saccharomyces cerevisiae, Candida sp., especially Candida albicans, Aspergillus fumigatus or Klebsiella pneumoniae in a sample for prognosing the status of a tumor patient.
